(19) United States
(12) Patent Application Publication
(10) Pub. No.: US 2008/0145268 A1
(43) Pub. Date: Jun. 19, 2008

(54) Deodorizing container that includes an anthraquinone ink

(76) Inventors: Stephanie M. Martin, Woodstock, GA (US); Jeffrey E. Fish, Dacula, GA (US); Kelly D. Arehart, Roswell, GA (US); Bao Trong Do, Decatur, GA (US); J. Gavin MacDonald, Decatur, GA (US)

Correspondence Address: DORITY & MANNING, P.A., POST OFFICE BOX 1449, GREENVILLE, SC, 29602-1449, US

(21) Appl. No.: 11/640,460

(22) Filed: Dec. 15, 2006

(51) Int. Cl. A61L 9/00; A62B 7/08

(52) U.S. Cl. 422/5; 422/122

(57) ABSTRACT

A deodorizing container for the disposal of absorbent articles, such as sanitary napkins, diapers, wipes, etc., is provided. More specifically, the container includes an odor control ink that contains an odor-reducing anthraquinone that is capable of interacting with malodorous compounds commonly associated with biological fluids (e.g., menses, urine, etc.).

### BACKGROUND OF THE INVENTION

Absorbent feminine care articles, such as sanitary napkins, panty containers, labial pads, and other types of catamenial devices, are used to absorb menses and other body fluids. These absorbent products are used during a women's menstrual cycle or between menstrual cycles for light incontinence purposes. Regardless, the absorbent articles are primarily designed for a single use, after which they are discarded into a toilet pail or trash receptacle. Unfortunately, however, storage in a toilet pail located in a bathroom or in some other trash receptacle may rapidly result in the development of disagreeable odors. As such, a need currently exists for a method for reducing the odor produced by personal care absorbent articles, particularly after they are disposed.

### SUMMARY OF THE INVENTION

In accordance with one embodiment of the present invention, a deodorizing container is disclosed that comprises a resiliently deformable substrate having an inner surface and an outer surface that define therebetween an interior space. An ink is present on the inner surface of the substrate that comprises an odor-reducing anthraquinone having the following general formula:

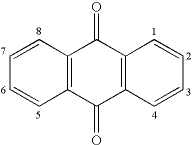

wherein the numbers 1 through 8 refer to optional substitution positions for functional groups.

In accordance with another embodiment of the present invention, a method for reducing the odor associated with an article that contains a bodily fluid (e.g., urine, menses, etc.) is disclosed. The comprises disposing the article (e.g., personal care absorbent article) into an interior space defined between an inner surface an outer surface of a deodorizing container. An ink is present on the inner surface of the container that comprises an odor-reducing anthraquinone configured to interact with a malodorous compound associated with the bodily fluid.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a perspective view of a deodorizing container that may be formed in accordance with one embodiment of the present invention, shown in an open configuration; and

FIG. 2 shows the deodorizing container of FIG. 1 in a closed configuration.

Repeat use of references characters in the present specification and drawings is intended to represent same or analogous features or elements of the invention.

### DETAILED DESCRIPTION OF REPRESENTATIVE EMBODIMENTS

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

### Definitions

As used herein, the term “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth; medical absorbent articles, such as garments, fenestration materials, underpads, bedpads, bandages, absorbent drapes, and medical wipes; food service wipers; clothing articles; and so forth. Various absorbent article configurations are described, for instance, in U.S. Pat. No. 5,649,916 to DiPalma, et al.; U.S. Pat. No. 6,110,158 to Kielpikowski; U.S. Pat. No. 6,663,611 to Blaney. et al.; U.S. Pat. No. 4,886,512 to Damico et al.; U.S. Pat. No. 5,558,659 to Sherrod et al.; U.S. Pat. No. 6,888,044 to Fell et al.; and U.S. Pat. No. 6,511,465 to Freiburger et al., as well as U.S. Patent Application Publication No. 2004/0060112 A1 to Fell et al., all of which are incorporated herein in their entirety by reference thereto for all purposes.

As used herein, the term “nonwoven web” refers to a web having a structure of individual fibers that are randomly interlaid, not in an identifiable manner as in a knitted fabric. Nonwoven webs include, for example, meltblown webs, spunbond webs, carded webs, wet-laid webs, airlaid webs, coform webs, hydraulically entangled webs, etc. The basis weight of the nonwoven web may generally vary, but is typically from about 5 grams per square meter (“gsm”) to 200 gsm, in some embodiments from about 10 gsm to about 150 gsm, and in some embodiments, from about 15 gsm to about 100 gsm.

As used herein, the term “meltblown” web or layer generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin, et al.; U.S. Pat. No. 4,307,143 to Meitner, et al.; and U.S. Pat. No. 4,707,398 to Wisneski, et al., which are incorporated herein in their entirety by reference thereto for all purposes. Meltblown fibers may be substantially continuous or discontinuous, and are generally tacky when deposited onto a collecting surface.

As used herein, the term “spunbond” web or layer generally refers to a nonwoven web containing small diameter substantially continuous filaments. The filaments are formed by extruding a molten thermoplastic material from a plurality of fine, usually circular, capillaries of a spinnerette with the diameter of the extruded filaments then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are incorporated herein in their entirety by reference thereto for all purposes. Spunbond filaments are generally not tacky when they are deposited onto a collecting surface. Spunbond filaments may sometimes have diameters less than about 40 micrometers, and are often between about 5 to about 20 micrometers.

### DETAILED DESCRIPTION

Generally speaking, the present invention is directed to a deodorizing container for the disposal of absorbent articles, such as sanitary napkins, diapers, wipes, etc. More specifically, the container includes an odor control ink that includes an odor-reducing anthraquinone having the following general formula:

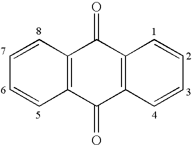

The numbers 1-8 shown in the general formula represent a location on the fused ring structure at which substitution of a functional group may occur. Some examples of such functional groups that may be substituted on the fused ring structure include halogen groups (e.g., chlorine or bromine groups), sulfonyl groups (e.g., sulfonic acid salts), alkyl groups, benzyl groups, amino groups (e.g., primary, secondary, tertiary, or quaternary amines), carboxy groups, cyano groups, hydroxy groups, phosphorous groups, etc. Functional groups that result in an ionizing capability are often referred to as “chromophores.” Substitution of the ring structure with a chromophore causes a shift in the absorbance wavelength of the compound. Thus, depending on the type of chromophore (e.g., hydroxyl, carboxyl, amino, etc.) and the extent of substitution, a wide variety of anthraquinones may be formed with varying colors and intensities. Other functional groups, such as sulfonic acids, may also be used to render certain types of compounds (e.g., higher molecular weight anthraquinones) water-soluble.

Anthraquinones may be classified for identification by their Color Index (CI) number, which is sometimes called a “standard.” For instance, some suitable anthraquinones that may be used in the present invention, as classified by their “CI” number, include Acid Black 48, Acid Blue 25 (D&C Green No. 5), Acid Blue 40, Acid Blue 41, Acid Blue 45, Acid Blue 80, Acid Blue 129, Acid Green 25, Acid Green 27, Acid Green 41, Acid Violet 43, Mordant Red 11 (Alizarin), Mordant Black 13 (Alizarin Blue Black B), Mordant Red 3 (Alizarin Red S), Mordant Violet 5 (Alizarin Violet 3R), Alizarin Complexone, Natural Red 4 (Carminic Acid), Disperse Blue 1, Disperse Blue 3, Disperse Blue 14, Natural Red 16 (Purpurin), Natural Red 8, Reactive Blue 2 (Procion Blue HB), Reactive Blue 19 (Remazol Brilliant Blue R); and so forth. The structures of Acid Blue 25, Acid Green 41, Acid Blue 45, Mordant Violet 5, Acid Blue 129, Acid Green 25, and Acid Green 27 are set forth below:

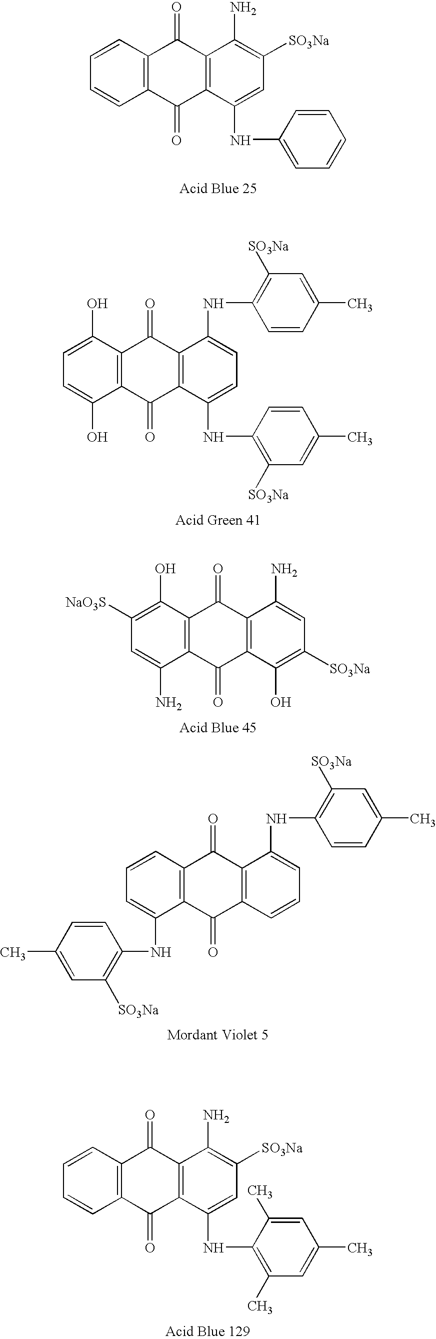

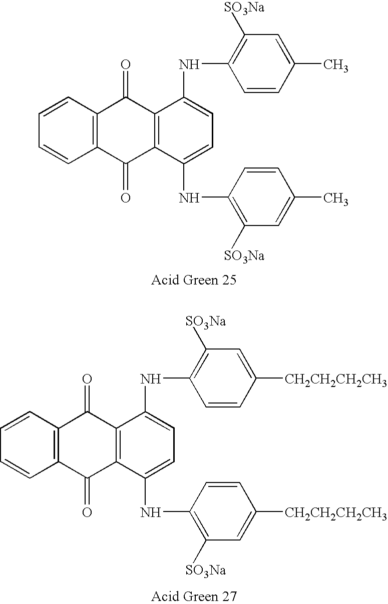

Without intending to be limited by theory, it is believed that the odor caused by many compounds is eliminated by the transfer of electrons to and/or from the malodorous compound. Specifically, oxidation of malodorous compounds via a reduction/oxidation (“redox”) reaction is believed to inhibit the production of the characteristic odor associated therewith. The discovery that certain anthraquinones are able to eliminate odor is believed to be due to their ability to function as an oxidizing agent in a redox reaction. Many common odorous compounds are capable of oxidizing (i.e., donate electrons) via a redox reaction. For instance, odorous compounds may include mercaptans (e.g., ethyl mercaptan), ammonia, amines (e.g., trimethylamine (TMA), triethylamine (TEA), etc.), sulfides (e.g., hydrogen sulfide, dimethyl disulfide (DMDS), etc.), ketones (e.g., 2-butanone, 2-pentanone, 4-heptanone, etc.) carboxylic acids (e.g., isovaleric acid, acetic acid, propionic acid, etc.), aldehydes, terpenoids, hexanol, heptanal, pyridine, and so forth. Upon oxidation, the odors associated with such compounds are often eliminated or at least lessened. It is also believed that the reduction of the anthraquinone via the redox reaction is readily reversible, and thus the reduced anthraquinone may be re-oxidized by any known oxidizing agent (e.g., oxygen, air, etc.). The reduction/oxidation reactions are rapid and may take place at room temperature. Thus, although the odor control mechanism may consume the anthraquinones, they may simply be regenerated by exposure to air. Thus, long-term odor control may be achieved without significantly affecting the ability of the anthraquinone to impart the desired color.

The ability of anthraquinones to accept electrons from another substance (i.e., be reduced) may be quantified using a technique known as redox potentiometry. Redox potentiometry is a technique that measures (in volts) the affinity of a substance for electrons—its electronegativity—compared with hydrogen (which is set at 0). Substances more strongly electronegative than (i.e., capable of oxidizing) hydrogen have positive redox potentials. Substances less electronegative than (i.e., capable of reducing) hydrogen have negative redox potentials. The greater the difference between the redox potentials of two substances (ΔE), the greater the vigor with which electrons will flow spontaneously from the less positive to the more positive (more electronegative) substance. As is well known in the art, redox potential may be measured using any of a variety of commercially available meters, such as an Oxidation Reduction Potential (ORP) tester commercially available from Hanna Instruments, Inc. of Woonsocket, R.I. The redox potential of the anthraquinones may, for instance, be less than about −50 millivolts (mV), in some embodiments less than about −150 mV, in some embodiments less than about −300 mV, and in some embodiments, less than about −500 mV. Although not always the case, the redox potential may vary based on the number and location of functional groups, such as sulfonic acid, on the anthraquinone structure. For example, 2-sulfonic acid anthraquinone has a redox potential of −380 mV; 2,6-disulfonic acid anthraquinone has a redox potential of −325 mV; and 2,7-disulfonic acid anthraquinone has a redox potential of −313 mV. The use of other functional groups may also have an affect on the ultimate redox potential of the compound. For example, Acid Blue 25, which also contains amino- and aramid functional groups, has a redox potential of −605 mV.

In addition to their ability to oxidize malodorous compounds, the chemical structure of certain anthraquinones may help improve odor elimination. For example, anthraquinones that have at least one unsubstituted ring may result in better odor inhibition than those that are substituted at each ring with a functional group. Interestingly, anthraquinones that are unsubstituted at the “first” ring (i.e., positions 5 through 8) appear to be particularly effective in reducing odor. Suitable examples of anthraquinones that are unsubstituted at locations at their first ring include, but are not limited to, Acid Blue 25, Acid Blue 129, Acid Green 25, and Acid Green 27, the structures of which are set forth above. Other exemplary odor control anthraquinones are described in U.S. Patent Application Publication No. 2005/0131363 to MacDonald. et al., which is incorporated herein in its entirety by reference thereto for all purposes.

The odor-reducing anthraquinone of the present invention may be used alone or in conjunction with other components. For example, nanoparticles may be employed in some embodiments that act as a carrier for the compound. The anthraquinone is believed to form a coordinate bond with an atom of certain nanoparticles (e.g., aluminum) via oxygen atoms present in the anthraquinone structure. As used herein, a “coordinate bond” refers to a shared pair of electrons between two atoms, wherein one atom supplies both electrons to the pair. When utilized, the amount of nanoparticles may generally vary in relation to the anthraquinone. For example, the molar ratio of the nanoparticles to the anthraquinone may range from about 10 to about 10,000, in some embodiments from about 50 to about 5,000, and in some embodiments, from about 100 to about 1,000.

The nanoparticles may possess various forms and shapes depending upon the desired result, such as a sphere, crystal, rod, disk, tube, string, etc. Regardless, the average particle size (e.g., diameter or width) of the nanoparticles is about 5 micrometers or less, in some embodiments about 1 micrometer or less, in some embodiments about 100 nanometers or less, in some embodiments from about 1 to about 50 nanometers, and in some embodiments, from about 2 to about 25 nanometers. Further, the surface area is typically about 50 square meters per gram (m²/g) or more, in some embodiments from about 100 m²/g to about 1000 m²/g, and in some embodiments, from about 150 m²/g to about 600 m²/g. Surface area may be determined by the physical gas adsorption (B.E.T.) method of Bruanauer, Emmet, and Teller, Journal of American Chemical Society, Vol. 60, 1938, p. 309, with nitrogen as the adsorption gas. Due to their small size and high surface area, it is believed that the nanoparticles provide an increased area for adsorbing malodorous compounds commonly associated with biological fluids (e.g., menses, urine, etc.). This may increase the statistical likelihood of contact between the anthraquinone and malodorous compounds, thereby enhancing the overall odor reduction provided by the ink. If desired, the nanoparticles may also be relatively nonporous or solid. That is, the nanoparticles may have a pore volume that is less than about 0.5 milliliters per gram (ml/g), in some embodiments less than about 0.4 milliliters per gram, in some embodiments less than about 0.3 ml/g, and in some embodiments, from about 0.2 ml/g to about 0.3 ml/g. Without intending to be limited by theory, it is believed that the solid nature, i.e., low pore volume, of the nanoparticles may enhance the uniformity and stability of the nanoparticles, without sacrificing their odor adsorption characteristics.

Any of a variety of nanoparticles may generally be employed in the present invention. One particularly suitable class of nanoparticles includes inorganic oxides, such as silica, alumina, zirconia, magnesium oxide, titanium dioxide, iron oxide, zinc oxide, copper oxide, zeolites, clays (e.g., smectite clay), combinations thereof, and so forth. Various examples of such nanoparticles are described in U.S. Patent Application Publication No. 2003/0203009 to MacDonald; U.S. Patent Application Publication No. 2005/0084412 to MacDonald, et al.; and U.S. Patent Application Publication No. 2005/0085144 to MacDonald, et al., which are incorporated herein in their entirety by reference thereto for all purposes. If desired, the nanoparticles may be selected to have a zeta potential that facilitates ionic bonding with certain compounds (e.g., anthraquinone, malodorous compounds, etc.), a substrate, and so forth. For example, the nanoparticles may possess a negative zeta potential, such as less than about 0 millivolts (mV), in some embodiments less than about −10 mV, and in some embodiments, less than about −20 mV. Examples of nanoparticles having a negative zeta potential include silica nanoparticles, such as Snowtex-C, Snowtex-O, Snowtex-PS, and Snowtex-OXS, which are available from Nissan Chemical of Houston, Tex. Alternatively, the nanoparticles may have a positive zeta potential, such as greater than about 0 millivolts, in some embodiments greater than about +20 millivolts (mV), in some embodiments greater than about +30 mV, and in some embodiments, greater than about +40 mV. The nanoparticles may, for instance, be formed entirely from a positively charged material, such as alumina. Examples of commercially available alumina nanoparticles include, for instance, Aluminasol 100, Aluminasol 200, and Aluminasol 520, which are available from Nissan Chemical Industries Ltd. The positive zeta potential may also be imparted by a continuous or discontinuous coating present on the surface of a core material. In one particular embodiment, for example, the nanoparticles are formed from silica nanoparticles coated with alumina. A commercially available example of such alumina-coated silica nanoparticles is Snowtex-AK, which is available from Nissan Chemical of Houston, Tex.

The odor control ink may also contain other components to facilitate application of the ink. For example, the ink may contain a binder for increasing the durability of the anthraquinone on the container, even when present at high levels. Suitable binders may include, for instance, those that become insoluble in water upon crosslinking. Crosslinking may be achieved in a variety of ways, including by reaction of the binder with a polyfunctional crosslinking agent. Examples of such crosslinking agents include, but are not limited to, dimethylol urea melamine-formaldehyde, urea-formaldehyde, polyamide epichlorohydrin, etc. In some embodiments, a polymer latex may be employed as the binder. The polymer suitable for use in the lattices typically has a glass transition temperature of about 30° C. or less so that the flexibility of the resulting container is not substantially restricted. Moreover, the polymer also typically has a glass transition temperature of about −25° C. or more to minimize the tackiness of the polymer latex. For instance, in some embodiments, the polymer has a glass transition temperature from about −15° C. to about 15° C., and in some embodiments, from about −10° C. to about 0° C. For instance, some suitable polymer lattices that may be utilized in the present invention may be based on polymers such as, but are not limited to, styrene-butadiene copolymers, polyvinyl acetate homopolymers, vinyl-acetate ethylene copolymers, vinyl-acetate acrylic copolymers, ethylene-vinyl chloride copolymers, ethylene-vinyl chloride-vinyl acetate terpolymers, acrylic polyvinyl chloride polymers, acrylic polymers, styrene-acrylic copolymers (e.g., Jonrez FV2080, available from MeadWestvaco Corporation, Charleston S.C.), nitrile polymers, and any other suitable anionic polymer latex polymers known in the art. The charge of the polymer lattices described above may be readily varied, as is well known in the art, by utilizing a stabilizing agent having the desired charge during preparation of the polymer latex.

Although polymer lattices may be effectively used as binders in the present invention, such compounds sometimes result in a reduction in drapability and an increase in residual odor. Thus, water-soluble organic polymers may also be employed as binders to alleviate such concerns. One class of water-soluble organic polymers found to be suitable in the present invention is polysaccharides and derivatives thereof. Polysaccharides are polymers containing repeated carbohydrate units, which may be cationic, anionic, nonionic, and/or amphoteric. In one particular embodiment, the polysaccharide is a nonionic, cationic, anionic, and/or amphoteric cellulosic ether. Suitable nonionic cellulosic ethers may include, but are not limited to, alkyl cellulose ethers, such as methyl cellulose and ethyl cellulose; hydroxyalkyl cellulose ethers, such as hydroxyethyl cellulose, hydroxypropyl cellulose, hydroxypropyl hydroxybutyl cellulose, hydroxyethyl hydroxypropyl cellulose, hydroxyethyl hydroxybutyl cellulose and hydroxyethyl hydroxypropyl hydroxybutyl cellulose; alkyl hydroxyalkyl cellulose ethers, such as methyl hydroxyethyl cellulose, methyl hydroxypropyl cellulose, ethyl hydroxyethyl cellulose, ethyl hydroxypropyl cellulose, methyl ethyl hydroxyethyl cellulose and methyl ethyl hydroxypropyl cellulose; and so forth. Suitable cellulosic ethers may include, for instance, those available from Akzo Nobel of Covington, Va. under the name “BERMOCOLL.” Still other suitable cellulosic ethers are those available from Shin-Etsu Chemical Co., Ltd. of Tokyo, Japan under the name “METOLOSE”, including METOLOSE Type SM (methycellulose), METOLOSE Type SH (hydroxypropylmethyl cellulose), and METOLOSE Type SE (hydroxyethylmethyl cellulose). One particular example of a suitable nonionic cellulosic ether is ethyl hydroxyethyl cellulose having a degree of ethyl substitution (DS) of 0.8 to 1.3 and a molar substitution (MS) of hydroxyethyl of 1.9 to 2.9. The degree of ethyl substitution represents the average number of hydroxyl groups present on each anhydroglucose unit that have been reacted, which may vary between 0 and 3. The molar substitution represents the average number of hydroxethyl groups that have reacted with each anhydroglucose unit. One such cellulosic ether is BERMOCOLL E 230FQ, which is an ethyl hydroxyethyl cellulose commercially available from Akzo Nobel. Other suitable cellulosic ethers are also available from Hercules, Inc. of Wilmington, Del. under the name “CULMINAL.”

The ink may also include various other components as is well known in the art, such as colorants, colorant stabilizers, photoinitiators, solvents, surfactants, humectants, biocides or biostats, electrolytic salts, pH adjusters, etc. For example, various components for use in an ink are described in U.S. Pat. No. 5,681,380 to Nohr, et al. and U.S. Pat. No. 6,542,379 to Nohr. et al., which are incorporated herein in their entirety by reference thereto for all purposes. Examples of suitable humectants include, for instance, ethylene glycol; diethylene glycol; glycerine; polyethylene glycol 200, 400, and 600; propane 1,3 diol; propylene-glycolmonomethyl ethers, such as Dowanol PM (Gallade Chemical Inc., Santa Ana, Calif.); polyhydric alcohols; or combinations thereof. Other additives may also be included to improve ink performance, such as a chelating agent to sequester metal ions that could become involved in chemical reactions over time, a corrosion inhibitor to help protect metal components of the printer or ink delivery system, a biocide or biostat to control unwanted bacterial, fungal, or yeast growth in the ink, and a surfactant to adjust the ink surface tension.

To form the odor control ink, its components may be initially dissolved or dispersed in a solvent. For example, one or more of the above-mentioned components may be mixed with a solvent, either sequentially or simultaneously, to form an ink that may be easily applied to a substrate. Any solvent capable of dispersing or dissolving the components is suitable, for example water; alcohols such as ethanol or methanol; dimethylformamide; dimethyl sulfoxide; hydrocarbons such as pentane, butane, heptane, hexane, toluene and xylene; ethers such as diethyl ether and tetrahydrofuran; ketones and aldehydes such as acetone and methyl ethyl ketone; acids such as acetic acid and formic acid; and halogenated solvents such as dichloromethane and carbon tetrachloride; as well as mixtures thereof. The concentration of solvent in the ink is generally high enough to allow easy application, handling, etc. If the amount of solvent is too large, however, the amount of anthraquinone deposited on the container might be too low to provide the desired odor reduction. Although the actual concentration of solvent employed will generally depend on the type of anthraquinone and the substrate on which it is applied, it is nonetheless typically present in an amount from about 40 wt. % to about 99 wt. %, in some embodiments from about 50 wt. % to about 95 wt. %, and in some embodiments, from about 60 wt. % to about 90 wt. % of the ink (prior to drying).

The solids content and/or viscosity of the ink may be varied to achieve the extent of odor reduction desired. For example, the ink may have a solids content of from about 5% to about 90%, in some embodiments from about 10% to about 80%, and in some embodiments, from about 20% to about 70%. By varying the solids content of the ink, the presence of the anthraquinone and other components in the odor control ink may be controlled. For example, to form an odor control ink with a higher level of a anthraquinone, the ink may be provided with a relatively high solids content so that a greater percentage of the anthraquinone is incorporated into the odor control ink during the application process. Generally, the viscosity is less than about 2×10⁶ centipoise, in some embodiments less than about 2×10⁵ centipoise, in some embodiments less than about 2×10⁴ centipoise, and in some embodiments, less than about 2×10³ centipoise, such as measured with a Brookfield viscometer, type DV-I or LV-IV, at 60 rpm and 20° C. If desired, thickeners or other viscosity modifiers may be employed in the ink to increase or decrease viscosity.

Once formed, the odor control ink of the present invention may be applied to the inner surface of a resiliently deformable substrate used to form the container so that it is able to readily contact malodorous compounds associated with any absorbent article disposed within the container. One benefit imparted by the odor control ink of the present invention is that it generally forms a durable coating that is not readily removed from the substrate upon application. Thus, the substrate may be pre-treated with the ink and subsequently incorporated into the container without substantial loss of its odor control properties. This allows the ink to more readily applied to certain areas of the container (e.g., inside surface) without substantial difficulty. Of course, it should be understood that the odor control ink may also be applied to the substrate after it is incorporated into the container.

Typically, the resiliently deformable substrate used to form the container is generally liquid-impermeable so that it is able to inhibit leakage of bodily fluids therefrom. The substrate may include one or multiple layers, and may be constructed from a variety of materials, such as films, nonwoven webs, paper webs, foams, etc. In one particular embodiment, the substrate includes a generally liquid impermeable film formed from a thermoplastic polymer, such as polyolefins (e.g., polyethylene, polypropylene, etc.), ethylene vinyl acetate, ethylene ethyl acrylate, ethylene acrylic acid, ethylene methyl acrylate, ethylene normal butyl acrylate, nylon, ethylene vinyl alcohol, polystyrene, polyurethane, and so forth. Such films may be mono- or multi-layered. The thickness of the film may vary depending upon the desired use. In most embodiments, however, the film has a thickness of about 50 micrometers or less, in some embodiments from about 1 to about 40 micrometers, in some embodiments from about 2 to about 35 micrometers, and in some embodiments, from about 5 to about 30 micrometers.

In addition to film forming polymer(s), other additives may also be incorporated into the film, such as melt stabilizers, processing stabilizers, heat stabilizers, light stabilizers, antioxidants, heat aging stabilizers, whitening agents, antiblocking agents, bonding agents, lubricants, fillers, etc. Fillers, for example, are particulates or other forms of material that may be added to the film polymer extrusion blend and that will not chemically interfere with the extruded film, but which may be uniformly dispersed throughout the film. Fillers may serve a variety of purposes, including enhancing film opacity and/or breathability (i.e., vapor-permeable and substantially liquid-impermeable). For instance, filled films may be made breathable by stretching, which causes the polymer to break away from the filler and create microporous passageways. Breathable microporous elastic films are described, for example, in U.S. Pat. Nos. 5,997,981; 6,015,764; and 6,111,163 to McCormack, et al.; U.S. Pat. No. 5,932,497 to Morman, et al.; U.S. Pat. No. 6,461,457 to Taylor, et al., which are incorporated herein in their entirety by reference thereto for all purposes. Further, hindered phenols are commonly used as an antioxidant in the production of films. Some suitable hindered phenols include those available from Ciba Specialty Chemicals under the trade name “Irganox®”, such as Irganox® 1076, 1010, or E 201. Moreover, bonding agents may also be added to the film to facilitate bonding of the film to additional materials (e.g., nonwoven webs). Examples of such bonding agents include hydrogenated hydrocarbon resins. Other suitable bonding agents are described in U.S. Pat. No. 4,789,699 to Kieffer et al. and U.S. Pat. No. 5,695,868 to McCormack, which are incorporated herein in their entirety by reference thereto for all purposes.

Besides films, the substrate may also contain a nonwoven web, such as spunbond webs, meltblown webs, bonded carded webs, air-laid webs, coform webs, hydraulically entangled webs, and so forth. Nonwoven webs may be formed by a variety of different materials. For instance, suitable polymers for forming nonwoven webs may include polyolefins, polyamides, polyesters, polycarbonates, polystyrenes, thermoplastic elastomers, fluoropolymers, vinyl polymers, and blends and copolymers thereof. Suitable polyolefins include, but are not limited to, polyethylene, polypropylene, polybutylene, and so forth; suitable polyamides include, but are not limited to, nylon 6, nylon 6/6, nylon 10, nylon 12 and so forth; and suitable polyesters include, but are not limited to, polyethylene terephthalate, polybutylene terephthalate, polytrimethyl terephthalate, polylactic acid, and so forth. Particularly suitable polymers for use in the present invention are polyolefins including polyethylene, for example, linear low density polyethylene, low density polyethylene, medium density polyethylene, and high density polyethylene; polypropylene; polybutylene; as well as copolymers and blends thereof.

The fibers used to form the nonwoven web may be in the form of substantially continuous fibers, staple fibers, and so forth. Substantially continuous fibers, for example, may be produced by known nonwoven extrusion processes, such as, for example, known solvent spinning or melt-spinning processes. In one embodiment, the nonwoven web contains substantially continuous melt-spun fibers formed by a spunbond process. The spunbond fibers may be formed from any melt-spinnable polymer, co-polymers or blends thereof. The denier of the fibers used to form the nonwoven web may also vary. For instance, in one particular embodiment, the denier of polyolefin fibers used to form the nonwoven web is less than about 6, in some embodiments less than about 3, and in some embodiments, from about 1 to about 3.

Nonwoven laminates may also be employed in some embodiments of the present invention. In one embodiment, the nonwoven laminate contains a meltblown layer positioned between two spunbond layers to form a spunbond/meltblown/spunbond (“SMS”) laminate. Various techniques for forming SMS laminates are described in U.S. Pat. No. 4,041,203 to Brock et al.; U.S. Pat. No. 5,213,881 to Timmons, et al.; U.S. Pat. No. 5,464,688 to Timmons. et al.; U.S. Pat. No. 4,374,888 to Bornslaeger; U.S. Pat. No. 5,169,706 to Collier. et al.; and U.S. Pat. No. 4,766,029 to Brock et al., as well as U.S. Patent Application Publication No. 2004/0002273 to Fitting. et al., all of which are incorporated herein in their entirety by reference thereto for all purposes. Of course, the nonwoven laminate may have other configuration and possess any desired number of meltblown and spunbond layers, such as spunbond/meltblown/meltblown/spunbond laminates (“SMMS”), spunbond/meltblown laminates (“SM”), etc.

If desired, the substrate may be subjected to one or treatments that enhance the resulting durability of the odor control ink. For instance, because the odor control ink may be aqueous-based, the substrate may be subjected to a hydrophilic treatment to improve its affinity for the ink. For example, the substrate may be subjected to corona field that results in morphological and chemical modifications of the surface of the substrate. The term “corona field” generally refers to a corona field of ionized gas. The dose or energy density to which the substrate is exposed may range from about 1 to about 500 watt-minute per square foot (w-min/ft²), in some embodiments from about 15 to about 350 w-min/ft², and in some embodiments, from about 20 to about 80 w-min/ft². The corona field may be applied to the substrate under ambient temperature and pressure; however, higher or lower temperature and pressures may be used. Various suitable corona discharge treatments are described, for instance, in U.S. Pat. No. 4,283,291 to Lowther; U.S. Pat. No. 3,754,117 to Walter; U.S. Pat. No. 3,880,966 to Zimmerman, et al.; and U.S. Pat. No. 3,471,597 to Schirmer, which are incorporated herein in their entirety by reference thereto for all purposes. In addition to or in conjunction with corona discharge treatment, the substrate may also be applied with a hydrophilic compound. One class of suitable hydrophilic compounds includes polysaccharides, such as described above.

Any of a variety of techniques may be employed in the present invention to apply the odor control ink to the substrate. For instance, the ink may be applied using rotogravure or gravure printing, either direct or indirect (offset). Gravure printing encompasses several well-known engraving techniques, such as mechanical engraving, acid-etch engraving, electronic engraving and ceramic laser engraving. Such printing techniques provide excellent control of the composition distribution and transfer rate. Gravure printing may provide, for example, from about 10 to about 1000 deposits per lineal inch of surface, or from about 100 to about 1,000,000 deposits per square inch. Each deposit results from an individual cell on a printing roll, so that the density of the deposits corresponds to the density of the cells. A suitable electronic engraved example for a primary delivery zone is about 200 deposits per lineal inch of surface, or about 40,000 deposits per square inch. By providing such a large number of small deposits, the uniformity of the deposit distribution may be enhanced. Suitable gravure printing techniques are also described in U.S. Pat. No. 6,231,719 to Garvey, et al., which is incorporated herein in its entirety by reference thereto for all purposes. Moreover, besides gravure printing, it should be understood that other printing techniques, such as flexographic printing, may also be used to apply the ink.

Still another suitable contact printing technique that may be utilized in the present invention is “screen printing.” Screen printing is performed manually or photomechanically. The screens may include a silk or nylon fabric mesh with, for instance, from about 40 to about 120 openings per lineal centimeter. The screen material is attached to a frame and stretched to provide a smooth surface. The stencil is applied to the bottom side of the screen, i.e., the side in contact with the substrate upon which the fluidic channels are to be printed. The ink is painted onto the screen, and transferred by rubbing the screen (which is in contact with the substrate) with a squeegee.

Ink-jet printing techniques may also be employed in the present invention. Ink-jet printing is a non-contact printing technique that involves forcing the ink through a tiny nozzle (or a series of nozzles) to form droplets that are directed toward the substrate. Two techniques are generally utilized, i.e., “DOD” (Drop-On-Demand) or “continuous” ink-jet printing. In continuous systems, ink is emitted in a continuous stream under pressure through at least one orifice or nozzle. The stream is perturbed by a pressurization actuator to break the stream into droplets at a fixed distance from the orifice. DOD systems, on the other hand, use a pressurization actuator at each orifice to break the ink into droplets. The pressurization actuator in each system may be a piezoelectric crystal, an acoustic device, a thermal device, etc. The selection of the type of ink jet system varies on the type of material to be printed from the print head. For example, conductive materials are sometimes required for continuous systems because the droplets are deflected electrostatically. Thus, when the sample channel is formed from a dielectric material, DOD printing techniques may be more desirable.

In addition to the printing techniques mentioned above, any other suitable application technique may be used in the present invention. For example, other suitable printing techniques may include, but not limited to, such as laser printing, thermal ribbon printing, piston printing, spray printing, flexographic printing, etc. Still other suitable application techniques may include bar, roll, knife, curtain, spray, slot-die, dip-coating, drop-coating, extrusion, stencil application, etc. Such techniques are well known to those skilled in the art.

The odor control ink may be cover an entire surface of the substrate, or it may be applied in a pattern. For example, the pattern may cover from about 10% to about 95%, in some embodiments from about 12% to about 90%, and in some embodiments, from about 15% to about 50% of the area of a surface of the substrate. The patterned application of the odor control ink may provide a variety of benefits, such as presenting a stark and highly visible contrast against a different color (e.g., the color of the background) and thus changing the overall appearance of the substrate. For example, the odor control ink may have a dark color and applied against a contrasting light background. Alternatively, a differently colored foreground may contrast with a dark background provided by the odor control ink. The relative degree of contrast between the odor control ink and the other color may be measured through a gray-level difference value. In a particular embodiment, the contrast may have a gray level value of about 45 on a scale of 0 to about 255, where 0 represents “black” and 255 represents “white.” The analysis method may be made with a Quantimet 600 Image Analysis System (Leica, Inc., Cambridge, UK). This system's software (QWIN Version 1.06A) enables a program to be used in the Quantimet User Interactive Programming System (QUIPS) to make the gray-level determinations. A control or “blank” white-level may be set using undeveloped Polaroid photographic film. An 8-bit gray-level scale may then be used (0-255) and the program allowed the light level to be set by using the photographic film as the standard. A region containing the other color (e.g., background or foreground) may then be measured for its gray-level value, followed by the same measurement of the ink. The routine may be programmed to automatically calculate the gray-level value of the odor control ink. The difference in gray-level value between the odor control ink and the other color may be about 45 or greater on a scale of 0-255, where 0 represents “black” and 255 represents “white.” The particular type or style of odor control ink pattern may include any arrangement of stripes, bands, dots, or other geometric shape. The pattern may include indicia (e.g., trademarks, text, and logos), floral designs, abstract designs, any configuration of artwork, etc.

The patterned application of odor control ink may also have various other functional benefits, including optimizing flexibility or some other characteristic of the substrate. The patterned application of odor control ink may provide different odor control properties to multiple locations of the substrate. For example, in one embodiment, the substrate may be treated with two or more regions of odor control ink that may or may not overlap. The regions may be on the same or different surfaces of the substrate. In one embodiment, one region of a substrate is coated with a first odor control ink, while another region is coated with a second odor control ink. If desired, one region may be configured to reduce one type of odor, while another region may be configured to reduce another type of odor. Alternatively, one region may possess a higher level of an odor control ink than another region or substrate to provide different levels of odor reduction.

Regardless of the method of application, the substrate may be dried at a certain temperature to drive the solvent from the odor control ink. For example, the substrate may be heated to a temperature of at least about 50° C., in some embodiments at least about 70° C., and in some embodiments, at least about 80° C. By minimizing the amount of solvent in the odor control ink, a larger amount of the anthraquinone may be available for contacting malodorous compounds, thereby enhancing odor reduction. It should be understood, however, that relatively small amounts of solvent may still be present. For example, the dried ink may contain a solvent in an amount less than about 10% by weight, in some embodiments less than about 5% by weight, and in some embodiments, less than about 1% by weight.

When dried, the relative percentages and solids add-on level of the resulting anthraquinone may vary to achieve the desired level of odor control. The “solids add-on level” is determined by subtracting the weight of the untreated substrate from the weight of the treated substrate (after drying), dividing this calculated weight by the weight of the untreated substrate, and then multiplying by 100%. One particular benefit of the present invention is that high solids add-on levels are achievable without a substantial sacrifice in durability of the ink and flexibility of the substrate. In some embodiments, for example, the add-on level of the ink is at least about 2%, in some embodiments from about 4% to about 40%, and in some embodiments, from about 6% to about 35%.

The concentration of the anthraquinone in the odor control ink is generally tailored to facilitate odor control without adversely affecting other properties of the resulting substrate, such as flexibility. The anthraquinone, for instance, may be present in the ink (after drying) in an amount of about 25 wt. % or more, in some embodiments from about 35 wt. % to about 98 wt. %, and in some embodiments, from about 50 wt. % to about 95 wt. %. Other components, such as a binder, may each be present in the ink (after drying) in an amount of from about 10 wt. % to about 80 wt. %, in some embodiments from about 20 wt. % to about 65 wt. %, and in some embodiments, from about 30 wt. % to about 50 wt. %.

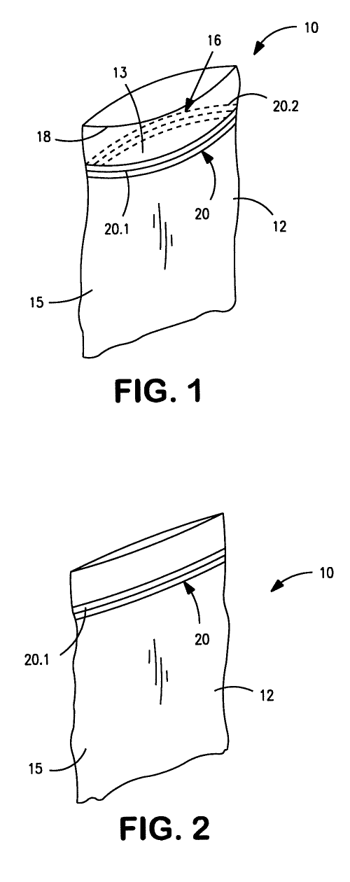

The configuration of the deodorizing container of the present invention is not particularly critical. Referring to FIGS. 1-2, for example, one particular embodiment of a re-sealable deodorizing container 10 is shown. In this embodiment, the container 10 contains a resiliently deformable substrate 12 (e.g., film) that includes an inner surface 13 and an outer surface 15, which define therebetween an interior space 16 and a mouth 18. Although not specifically depicted, the odor control ink of the present invention may be present on the inner surface 13 so that it is able to readily contact malodorous compounds associated with an absorbent article (not shown) contained in the space 16. If desired, the container 10 may also employ a closure mechanism to help provide sufficient containment of the odors evolving from an absorbent article so that adequate time exists for the malodorous compounds to interact with the odor control ink. In the illustrated embodiment, for instance, the container 10 includes a closure 20 for sealing the mouth 18 that includes releasably engageable complementary tongue and groove strips 20.1 and 20.2. The strip 20.2 of the closure 20 defines a groove formation and is disposed on the substrate 12 in register with the strip 20.1, which defines a complementary tongue formation. Upon disposing an absorbent article (e.g., sanitary napkin) in the container 10, the mouth 18 may thus be closed by securing the strips 20.1 and 20.2 to each other. Suitable closures may include, for instance, tongue-in-groove closures, such as Ziploc® (S.C. Johnson & Son, Inc.) and Zip-Pak® (Illinois Tool Works) closures. Besides tongue-in-groove closures, other mechanisms for sealing the container may also be employed in the present invention. For example, sliding seals (e.g., MiniGrip®, available from Illinois Tool Works), hook-and-loop fasteners (e.g., Velcro®, available from Velcro Industries B.V.), etc., may be employed as sealing mechanisms. In other embodiments, an adhesive (e.g., pressure-sensitive) may be employed as a sealing mechanism. Suitable pressure-sensitive adhesives, for instance, may include acrylic adhesives, natural rubber adhesives, tackified block copolymer adhesives, polyvinyl acetate adhesives, ethylene vinyl acetate adhesives, silicone adhesives, polyurethane adhesives, thermosettable pressure-sensitive adhesives, such as epoxy acrylate or epoxy polyester pressure-sensitive adhesives, etc.

The effectiveness of the deodorizing container of the present invention may be measured in a variety of ways. For example, the percent of a malodorous compound adsorbed by the odor control ink may be determined in accordance with the headspace gas chromatography test set forth herein. In some embodiments, for instance, the container is capable of adsorbing at least about 25%, in some embodiments at least about 45%, and in some embodiments, at least about 65% of a particular malodorous compound, such as mercaptans (e.g., ethyl mercaptan), ammonia, amines (e.g., trimethylamine (TMA), triethylamine (TEA), etc.), sulfides (e.g., hydrogen sulfide, dimethyl disulfide (DMDS), etc.), ketones (e.g., 2-butanone, 2-pentanone, 4-heptanone, etc.) carboxylic acids (e.g., isovaleric acid, acetic acid, propionic acid, etc.), aldehydes, terpenoids, hexanol, heptanal, pyridine, and so forth. The effectiveness of the ink in removing odors may also be measured in terms of “Relative Adsorption Efficiency”, which is determined using headspace gas chromatography and measured in terms of milligrams of odor adsorbed per gram of the ink. It should be recognized that the chemistry of any one type of odor control ink may not be suitable to reduce all types of malodorous compounds, and that low adsorption of one or more malodorous compounds may be compensated by good adsorption of other malodorous compounds.

The present invention may be better understood with reference to the following examples.

### EXAMPLE 1

Initially, the following five (5) solutions were formed:
1. An aqueous solution of 2.5% wt/wt D&C Green No. 25 (Sigma-Aldrich Chemical Co., St. Louis, Mo.).
2. An aqueous solution of 2.5% wt/wt D&C Green No. 25 (Sigma-Aldrich Chemical Co., St. Louis, Mo.) and 5.0% wt/wt Snowtex AK nanoparticles (Nissan Chemical America of Houston, Tex.).
3. An aqueous solution of 2% wt/wt BERMOCOLL E 230FQ (Akzo Nobel).
4. An aqueous solution of 17.8% wt/wt calcium carbonate.
5. An aqueous solution of 60% wt/wt of a white flexographic ink (Akzo Nobel).

From these solutions, odor control inks were formed as follows:

| | Solution | Solution | Solution | Solution | Solution |
|---|---|---|---|---|---|
| Ink | No. 1 | No. 2 | No. 3 | No. 4 | No. 5 |
| Sample | (mL) | (mL) | (mL) | (mL) | (mL) |
| A | — | 1.0 | 1.0 | — | 0.5 |
| B | 1.0 | — | 1.0 | — | 0.5 |
| C | — | 1.0 | 1.0 | 1.0 | — |
| D | 1.0 | — | 1.0 | 1.0 | — |
| E | — | 1.0 | 1.0 | — | — |
| F | 1.0 | — | 1.0 | — | — |

Once formed, an aliquot (1 mL) of each ink sample was pipetted onto a corona-treated polyethylene film (Pliant Corp. of Schaumburg, Ill.) in front of an unthreaded metal draw down bar. The bar was then pulled down by hand to draw down the fluid as smoothly as possible. The treated films were allowed to dry in air. Strips were cut from each test sheet and placed in small glass jars with a slice of garlic for overnight incubation at room temperature. The next day, the jars were assessed. It was determined that Sample B (containing D&C Green No. 5, E230 binder, and the flexographic ink) achieved the best odor reduction. The nanoparticles did not appear to provide a significant improvement in odor reduction. A drop of water was also placed onto each of the samples and wiped, which resulted in the removal of the ink for Samples C-F. The films were then placed in an oven to cure for 15 minutes at 80° C. After this time, a drop of water was also placed onto each of the samples and wiped, which again resulted in the removal of the ink for Samples C-F.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A deodorizing container comprising a resiliently deformable substrate having an inner surface and an outer surface that define therebetween an interior space, wherein an ink is present on the inner surface of the substrate that comprises an odor-reducing anthraquinone having the following general formula: 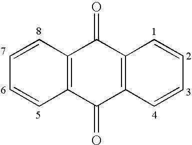
wherein the numbers 1 through 8 refer to optional substitution positions for functional groups.

2. The deodorizing container of claim 1, wherein the anthraquinone is substituted with halogen groups, alkyl groups, benzyl groups, amino groups, carboxy groups, cyano groups, hydroxy groups, phosphorous groups, sulfonic acid groups, or combinations thereof.

3. The deodorizing container of claim 1, wherein at least one ring of the anthraquinone is unsubstituted with functional groups.

4. The deodorizing container of claim 3, wherein positions 5 through 8 of the anthraquinone are unsubstituted with functional groups.

5. The deodorizing container of claim 1, wherein the odor-reducing anthraquinone is selected from the group consisting of Acid Blue 25, Acid Blue 40, Acid Blue 45, Acid Blue 80, Acid Blue 129, Acid Green 25, Acid Green 27, Acid Green 41, D&C Green No. 5, Mordant Violet 5, Mordant Black 13, Reactive Blue 19, and Reactive Blue 2.

6. The deodorizing container of claim 1, wherein the ink further comprises inorganic oxide nanoparticles.

7. The deodorizing container of claim 1, wherein the anthraquinone constitutes from about 25 wt. % or more of the ink.

8. The deodorizing container of claim 1, wherein the anthraquinone constitutes from about 35 wt. % to about 98 wt. % of the ink.

9. The deodorizing container of claim 1, wherein the ink further comprises a binder.

10. The deodorizing container of claim 9, wherein the binder constitutes from about 10 wt. % to about 80 wt. % of the ink.

11. The deodorizing container of claim 1, wherein the substrate includes a thermoplastic film.

12. The deodorizing container of claim 11, wherein a surface of the film is treated with a corona field, hydrophilic compound, or a combination thereof.

13. The deodorizing container of claim 1, wherein the substrate includes a nonwoven web.

14. The deodorizing container of claim 1, wherein the ink covers from about 10% to about 95% of the area of the inner surface of the substrate.

15. The deodorizing container of claim 1, wherein the ink covers substantially the entire inner surface of the substrate.

16. The deodorizing container of claim 1, further comprising a re-sealable closure.

17. The deodorizing container of claim 16, wherein the re-sealable closure includes a tongue-in-groove closure, sliding closure, hook-and-loop fastener, adhesive, or a combination thereof.

18. A disposable package comprising the deodorizing container of claim 1, wherein a personal care absorbent article is contained within the interior space defined by the substrate.

19. A method for reducing the odor associated with an article that contains a bodily fluid, the method comprising disposing the article into an interior space defined between an inner surface an outer surface of a deodorizing container, wherein an ink is present on the inner surface of the container that comprises an odor-reducing anthraquinone having the following general formula: 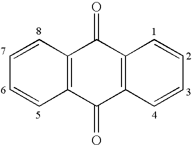
wherein the numbers 1 through 8 refer to optional substitution positions for functional groups, wherein the anthraquinone is configured to interact with a malodorous compound associated with the bodily fluid.

20. The method of claim 19, wherein the anthraquinone is substituted with halogen groups, alkyl groups, benzyl groups, amino groups, carboxy groups, cyano groups, hydroxy groups, phosphorous groups, sulfonic acid groups, or combinations thereof.

21. The method of claim 19, wherein at least one ring of the anthraquinone is unsubstituted with functional groups.

22. The method of claim 19, wherein positions 5 through 8 of the anthraquinone are unsubstituted with functional groups.

23. The method of claim 19, wherein the odor-reducing anthraquinone is selected from the group consisting of Acid Blue 25, Acid Blue 40, Acid Blue 45, Acid Blue 80, Acid Blue 129, Acid Green 25, Acid Green 27, Acid Green 41, D&C Green No. 5, Mordant Violet 5, Mordant Black 13, Reactive Blue 19, and Reactive Blue 2.

24. The method of claim 19, wherein the anthraquinone constitutes from about 25 wt. % or more of the ink.

25. The method of claim 19, wherein the anthraquinone constitutes from about 35 wt. % to about 98 wt. % of the ink.

26. The method of claim 19, wherein the ink further comprises a binder in an amount of from about 10 wt. % to about 80 wt. % of the ink.

27. The method of claim 19, wherein the container comprises a thermoplastic film on which the odor control ink is present.

28. The method of claim 19, wherein the container comprises a nonwoven web on which the odor control ink is present.

29. The method of claim 19, further comprising sealing the container after disposal of the article within the interior space.

30. The method of claim 19, wherein the article is a personal care absorbent article.

31. The method of claim 19, wherein the malodorous compound is a mercaptan, ammonia, amine, sulfide, ketone, carboxylic acid, aldehyde, terpenoid, hexanol, heptanal, pyridine, or a combination thereof.
